(19) United States
(12) Patent Application Publication
(10) Pub. No.: US 2004/0073199 A1
(43) Pub. Date: Apr. 15, 2004

(54) Method for alteration of iris pigment

(76) Inventor: Gregg S. Homer, Sherman Oaks, CA (US)

Correspondence Address: David A Farah, Sheldon & Mak, 9th Floor, 225 South Lake, Pasadena, CA, 91101, US

(21) Appl. No.: 10/466,963

(22) PCT Filed: Sep. 20, 2001
(86) PCT No.: PCT/US01/29481

(30) Foreign Application Priority Data
Feb. 2, 2001 (WO) PCT/US01/03569

(51) Int. Cl. A61B 18/18

(52) U.S. Cl. 606/4; 606/3; 606/5; 128/898

(57) ABSTRACT

A method for altering iris pigment in a human or animal, thereby altering iris color of an iris from a first iris color to a second iris color. The method comprises selecting one or more than one laser capable of generating one or more than one laser beam that will remove iris pigment of at least two different iris pigment colors from the iris and applying the one or more than one laser beam to the iris of a first iris color to remove the iris pigment. Removal of the iris pigment causes an alteration in the color of the first iris from the first iris color to the second iris color, where either the second iris color is both a different hue and less saturated than the first iris color, or where the second iris color is both a different hue and less saturated than the first iris color.

### CROSS-REFERENCE TO RELATED APPLICATION

The present Application claims the benefit of International Application PCT/US01/03569, filed Feb. 2, 2001, entitled “Method for Alteration of Iris Pigment,” and is a continuation in part of U.S. patent application Ser. No. 09/776,345, filed Feb. 2, 2001, entitled “Method for Alteration of Iris Pigmentation,” the contents of which are incorporated herein by reference in their entirety.

### BACKGROUND

Iris color in humans varies widely between ethnic groups and between individuals. Iris color of a normal human is dependent on the presence and density of colored pigment in pigment cells in the iris. The colored pigment exists in more than one form, for example, the brown pigment eumelanin and the yellow pigment pheomelanin. Iris pigment epithelial cells containing the pigment eumelanin are present in both the anterior layer of the iris and in the deeper superficial stroma of the iris, while iris pigment epithelial cells containing the pigment pheomelanin are primarily present in the superficial stroma of the iris.

A complete lack of iris pigment results in a pink color due to the coloration of vascular components of the iris stroma. Very low pigment density causes the scatter of white light passing through the iris and is as blue, a process similar to the scatter in the atmosphere resulting in a blue sky. The remaining iris colors result from the increasing density of one or more of the forms of iris pigment.

Iris color plays and has played a significant social function as an attribute of beauty. In recent years, alteration of some types of iris color has become possible through the use of colored contact lenses. Such colored contact lenses can have a prescriptive optical power, or can be optically neutral such that the lenses serves a cosmetic function only.

There are several disadvantages associated with the use of colored contact lenses for cosmetic purposes. First, the lenses have the same potential complications of use as contact lenses with prescriptive optical powers, including allergic reactions to the lens material and infections from improper handling. Further, contact lenses cannot be tolerated by some potential users due to the discomfort. Additionally, colored contact lenses require a degree of dexterity to insert and remove that is not possessed by all potential users. Further, permanent changes in iris color cannot be achieved through the use of colored contact lenses.

Another method for altering iris color involves the use of colored lens implanted anterior to the iris. Such implants require an invasive procedure to place the lens in position. Because of the potential complication of an invasive procedure and of leaving a foreign body within the eye, the implantation of color lenses has not become a widely adopted procedure.

Therefore, there remains a need for a method to alter iris pigment in a human which does not require colored contact lenses or implanted lenses. Additionally, there remains a need for a method to alter iris pigment in a human which can permanently alter iris color.

### SUMMARY

According to one embodiment of the present invention, there is provided a method for altering iris pigment in a human or animal, thereby altering iris color of a first iris from a first iris color to a second iris color. The method comprises, first, selecting one or more than one laser capable of generating one or more than one laser beam that will remove substantially similar amounts of iris pigment of at least two different iris pigment colors from the first iris, and then applying the one or more than one laser beam to the first iris of a first iris color to remove the iris pigment. The removal of the iris pigment causes an alteration in the color of the first iris from the first iris color to the second iris color. The second iris color is substantially the same hue as the first iris color but is less saturated than the first iris color, or the second iris color is both a different hue and less saturated than the first iris color.

In one embodiment, the human or animal is a human patient, and the method further comprises consulting with the patient to determine the second iris color before applying the one or more than one laser beam. In a preferred embodiment, the method further comprises repeating the selecting and applying steps at least one day after applying the one or more than one laser beam. In another preferred embodiment, the method further comprises repeating the selecting and applying steps at after a time between about 1 day and about 2 years after applying the one or more than one laser beam. In another preferred embodiment, the method further comprises repeating the selecting and applying steps at least one day after a time between about 1 week and about 1 month after applying the one or more than one laser beam.

In one embodiment, the second iris color does not naturally occur in a human iris. In another embodiment, the method further comprises creating an opening in the cornea of the human or animal before applying the one or more than one laser beam, and then applying the one or more than one laser beam through the opening.

In one embodiment, the one or more than one laser selected is a pulse dye laser. In another embodiment, the one or more than one laser beam generated has a wavelength of between about 300 nm and about 900 nm.

In a preferred embodiment, the method further comprises altering iris pigment in the human or animal, thereby altering iris color of a second iris from a third iris color to a fourth iris color. The method comprises selecting one or more than one laser capable of generating one or more than one laser beam that will remove iris pigment from the second iris, and then, applying the one or more than one laser beam to the second iris of the third iris color to remove iris pigment. The removal of the iris pigment of the second selected color causes an alteration in the color of the second iris from the third iris color to the fourth iris color. In a particularly preferred embodiment, the second iris color is substantially same as the fourth iris color. In another particularly preferred embodiment, the second iris color is substantially different than the fourth iris color.

### DESCRIPTION

According to one embodiment of the present invention, there is provided a method for altering iris pigment in a human, and thereby altering iris color from a first iris color to a second iris color. In one embodiment, the method comprises selecting one or more than one laser capable of generating one or more than one laser beam that will selectively remove iris pigment substantially of only a first pigment color, or that will remove iris pigment substantially uniformly of all iris pigment colors to decrease the overall density of all colors of iris pigment, or that will remove iris pigment of a first pigment color and a second pigment color while removing more iris pigment of the first pigment color than iris pigment of the second pigment color. Next, the one or more than one laser beam is applied to an iris of a first iris color to remove iris pigment. The removal of iris pigment causes an alteration in the color of the iris from the first iris color to the second iris color, where the second iris color is a different hue than the first iris color, or is a different saturation than the first iris color or is both a different hue and a different saturation than the first iris color. The method will now be explained in greater detail.

According to another embodiment of the present invention, there is provided a method for altering the iris pigment in the iris of an animal or a human to change the color of the iris from a first iris color to a second iris color. The method of the present invention is performed as follows. First, the human patient or animal is examined and, if a human patient, a determination is made in consultation with the human patient as to the patient's desired iris color. Next, a determination is made as to whether the patient's desired iris color can be obtained using the method of the present invention by virtue of laser alteration of the patient's existing iris pigment as will be understood by those in the art with reference to this disclosure. For example, if the patient has brown irises and desires to have green irises, then laser alteration of the patient's iris pigment using the present method can alter the patient's irises to green. Similarly, if the patient has brown irises and desires to less saturated brown irises, then laser alteration of the patient's iris pigment using the present method can alter the patient's irises to less saturated brown irises. Further, if the patient has brown irises and desires to have green irises that are also less saturated, then laser alteration of the patient's iris pigment using the present method can alter the patient's irises to less saturated green irises. If, however, the patient has blue irises and desires to have brown irises, then laser alteration of the patient's iris pigment using the present method cannot alter the patient's irises to brown, as will be understood by those in the art with reference to this disclosure.

Next, the method comprises selecting one or more than one laser capable of generating one or more than one laser beam. Depending on the first iris color and the desired second iris color, as will be understood by those in the art with reference to this disclosure, the one or more than one laser beam is selected that will either selectively remove iris pigment substantially of only a first selected pigment color, or that will remove iris pigment substantially uniformly of all iris pigment colors to decrease the overall density all colors of iris pigment, or that will remove iris pigment of a first pigment color and a second pigment color while removing more iris pigment of the first pigment color than iris pigment of the second pigment color.

For example, if the second iris color is the substantially the same hue but less saturated than the first iris color, than the one or more than one laser is selected that will remove iris pigment substantially uniformly of all iris pigment colors to decrease the overall density of all colors of iris pigment. Alternately, if the second iris color is a different hue than the first iris color, than the one or more than one laser is selected that will selectively remove iris pigment substantially of only a first selected pigment color. Similarly, if the second iris color is both a different hue and less saturated than the first iris color, than the one or more than one laser is selected that will remove iris pigment of a first pigment color and a second pigment color while removing more iris pigment of the first pigment color than iris pigment of the second pigment color, depending on the first iris color and the desired second iris color, as will be understood by those in the art with reference to this disclosure.

Next, the one or more than one laser beam is applied to the iris of the first iris color at a sufficient intensity and for a sufficient time to remove iris pigment, where the removal of the iris pigment causes an alteration in the color of the iris from the first iris color to the second iris color. If more than one laser beam is used, the laser beams can be applied sequentially or simultaneously. The second iris color is a different hue than the first iris color, or is substantially the same hue as the first iris color but less saturated than the first iris color, or is both a different hue and less saturated than the first iris color. Further, it is possible using methods according to the present invention to alter iris pigment of a patient to result in an iris color which is not naturally occurring, such as violet, or to alter iris pigment differently between two eyes of a patient to result in the patient having irises of different colors by differentially treating each iris of a patient according to one method of the present invention or by treating only one iris of the patient according to the present invention.

Preferably, the intensity of the laser beam is set to a level that minimizes the damage to any ocular tissue while still allowing satisfactory removal of the required iris pigment. The method of the present invention can further include creating an opening in the cornea of the human before applying the one or more than one laser beam, and then applying the one or more than one laser beam through the opening in order to minimize damage to the anterior ocular structures. If necessary, a temporary contact lens can be applied to reduce post-procedure discomfort.

Additionally, the method of the present invention can be repeated at a time spaced apart from the original application of the one or more than one laser, in order to further alter the iris pigment, after allowing the iris and associated tissue to heal from the original application. For example, the method can be repeated at least one day after applying the one or more than one laser beam to the iris. In a preferred embodiment, the method is repeated at a time between about 1 day and about 2 years after applying the one or more than one laser beam to the iris. In a particularly preferred embodiment, the method is repeated at a time between about 1 week and about 1 month after applying the one or more than one laser beam to the iris.

The use of lasers to remove iris pigment is advantageous because specific lasers can be selected which create laser beams which are selectively absorbed by iris pigment of specific colors. This property allows the selection of lasers to selectively destroy iris pigment of specific colors while not destroying iris pigment of other colors. For example, the one or more than one laser beam generated could be selected to substantially remove only eumelanin or substantially remove only pheomelanin. Alternately, one or more than one laser beam can be selected to remove iris pigment substantially uniformly of all iris pigment colors to decrease the overall density all colors of iris pigment. Additionally, the one or more than one laser beam can be selected to remove iris pigment of a first pigment color and a second pigment color while removing more iris pigment of the first pigment color than iris pigment of the second pigment color.

In a preferred embodiment, the one or more than one laser selected is a pulse dye lasers because the wavelength of the laser beam is determined by the color of the material through which the laser beams pass. This advantageously increases the specificity of iris pigment destruction by the laser beams by selecting appropriate dyes. Moreover, the intensity of the laser beams can be adjusted to minimize damage to ocular tissue and iris pigments that are not specifically targeted. In another preferred embodiment, the one or more than one laser is a pulse dye laser and the wavelength generated by the one or more than one laser is between about 300 nm and about 900 nm. For example, suitable lasers includes the Due-220, DUO-221, and the DUO-210 models, as well as the DYE 120, the DYE 121, and the DYE 110 models (Laser Science, Inc., Franklin, Mass. US).

In another preferred embodiment, the one or more than one laser selected is a modified excimer laser which permits the operator to alter the nature and quantity of the material through which the laser beam passes. Preferably, the one or more than one excimer laser will generate a wavelength of between about 100 nm and 250 nm. For example, the ArF excimer laser used in ophthalmic photoablation produces a beam of ultraviolet light with a wavelength of 193 nm. The ArF excimer laser beam passes though a specified quantity of argon fluoride, and both the material and its quantity can be modified to achieve the desired wavelength. Additionally, LaserSight Technologies, Inc. (Winter Park, Fla. US) has devised two solid state lasers: the LaserHarmonic-1 and LaserHarmonic-2. The LaserHarmonic-1 is flash lamp pumped and employs the fifth harmonic of a Nd:Yag at 213 nm. The LaserHarmonic-2 is a diode pumped fifth harmonic Nd:YLF laser at 209 nm. While a pulse dye laser or an excimer laser are preferred, other lasers can be used in the present method.

### EXAMPLE I

One method of the present invention is performed as follows. An adult male patient is selected who desires to change his brown iris color to green. He is found in satisfactory general and ocular health. After being counseled regarding the procedure and being prepared, a suitable laser is selected and is applied to each iris through the patient's intact corneas to remove pigment present in his irises until the remaining iris pigment is to render his irises green.

Although the present invention has been discussed in considerable detail with reference to certain preferred embodiments, other embodiments are possible. Therefore, the scope of the appended claims should not be limited to the description of preferred embodiments contained in this disclosure.

I claim:

1. A method for altering iris pigment in a human or animal, thereby altering iris color of a first iris from a first iris color to a second iris color, the method comprising:
a) selecting one or more than one laser capable of generating one or more than one laser beam that will remove substantially similar amounts of iris pigment of at least two different iris pigment colors from the first iris; and
b) applying the one or more than one laser beam to the first iris of a first iris color to remove the iris pigment;
where the removal of the iris pigment causes an alteration in the color of the first iris from the first iris color to the second iris color; and
where the second iris color is substantially the same hue as the first iris color but where the second iris color is less saturated than the first iris color.

2. The method of claim 1, where the human or animal is a human patient, and where the method further comprises consulting with the patient to determine the second iris color before applying the one or more than one laser beam.

3. The method of claim 1, further comprising repeating steps a) and b) at least one day after applying the one or more than one laser beam.

4. The method of claim 1, further comprising repeating steps a) and b) after a time between about 1 day and about 2 years after applying the one or more than one laser beam.

5. The method of claim 1, further comprising repeating steps a) and b) after a time between about 1 week and about 1 month after applying the one or more than one laser beam.

6. The method of claim 1, where the second iris color does not naturally occur in a human iris.

7. The method of claim 1, further comprising creating an opening in the cornea of the human or animal before applying the one or more than one laser beam, and then applying the one or more than one laser beam through the opening.

8. The method of claim 1, where the one or more than one laser selected is a pulse dye laser.

9. The method of claim 1, where the one or more than one laser beam generated has a wavelength of between about 300 nm and about 900 nm.

10. The method of claim 1, further comprising altering iris pigment in the human or animal, thereby altering iris color of a second iris from a third iris color to a fourth iris color, the method comprising:
a) selecting one or more than one laser capable of generating one or more than one laser beam that will remove iris pigment from the second iris; and
b) applying the one or more than one laser beam to the second iris of the third iris color to remove iris pigment; and
where the removal of the iris pigment of the second selected color causes an alteration in the color of the second iris from the third iris color to the fourth iris color.

11. The method of claim 10, where the second iris color is substantially same as the fourth iris color.

12. The method of claim 10, where the second iris color is substantially different than the fourth iris color.

13. A method for altering iris pigment in a human or animal, thereby altering iris color of a first iris from a first iris color to a second iris color, the method comprising:
a) selecting one or more than one laser capable of generating one or more than one laser beam that will remove iris pigment of at least two different iris pigment colors from the first iris; and
b) applying the one or more than one laser beam to the first iris of a first iris color to remove the iris pigment;
where the removal of the iris pigment causes an alteration in the color of the first iris from the first iris color to the second iris color; and
where the second iris color is both a different hue and less saturated than the first iris color.

14. The method of claim 13, where the human or animal is a human patient, and where the method further comprises consulting with the patient to determine the second iris color before applying the one or more than one laser beam.

15. The method of claim 13, further comprising repeating steps a) and b) at least one day after applying the one or more than one laser beam.

16. The method of claim 13, further comprising repeating steps a) and b) after a time between about 1 day and about 2 years after applying the one or more than one laser beam.

17. The method of claim 13, further comprising repeating steps a) and b) after a time between about 1 week and about 1 month after applying the one or more than one laser beam.

18. The method of claim 13, where the second iris color does not naturally occur in a human iris.

19. The method of claim 13, further comprising creating an opening in the cornea of the human or animal before applying the one or more than one laser beam, and then applying the one or more than one laser beam through the opening.

20. The method of claim 13, where the one or more than one laser selected is a pulse dye laser.

21. The method of claim 13, where the one or more than one laser beam generated has a wavelength of between about 300 nm and about 900 nm.

22. The method of claim 13, further comprising altering iris pigment in the human or animal, thereby altering iris color of a second iris from a third iris color to a fourth iris color, the method comprising:
a) selecting one or more than one laser capable of generating one or more than one laser beam that will remove iris pigment from the second iris; and
b) applying the one or more than one laser beam to the second iris of the third iris color to remove iris pigment; and
where the removal of the iris pigment of the second selected color causes an alteration in the color of the second iris from the third iris color to the fourth iris color.

23. The method of claim 22, where the second iris color is substantially same as the fourth iris color.

24. The method of claim 22, where the second iris color is substantially different than the fourth iris color.
